(19) United States
(12) Patent Application Publication
(10) Pub. No.: US 2016/0200665 A1
(43) Pub. Date: Jul. 14, 2016

(54) SYNTHESIS OF BIPHENYLALANINOL VIA NOVEL INTERMEDIATES

(71) Applicant: DPX HOLDINGS B.V., Amsterdam (NL)

(72) Inventors: Axel Zimmermann, Echt (NL); Petrus Johannes Hermsen, Echt (NL); Martin Helmut Friedrich Hanbauer, Echt (NL)

(21) Appl. No.: 14/912,313

(22) PCT Filed: Aug. 21, 2014
(86) PCT No.: PCT/EP2014/067804

§ 371 Date: Feb. 16, 2016

(30) Foreign Application Priority Data
Aug. 21, 2013 (EP) 13181136.6

(51) Int. Cl. C07C 215/28; C07C 271/16; C07C 269/04; C07C 231/12; C07C 213/02

(52) U.S. Cl. CPC C07C 215/28; C07C 231/12; C07C 213/02; C07C 269/04; C07C 271/16; C07B 2200/07

(57) ABSTRACT

The invention relates to a novel synthesis route towards R-biphenylalaninol and to the intermediates applied in this synthesis route. The process according to the invention and the intermediate compounds are useful in the synthesis of pharmaceutically active compounds.

The invention relates to a novel synthesis route towards R-biphenylalaninol and to the intermediates applied in this synthesis route. The process according to the invention and the intermediate compounds are useful in the synthesis of pharmaceutically active compounds.

### BACKGROUND OF THE INVENTION

The present invention relates to methods to prepare N-boc protected R-biphenylalaninol, which is a key intermediate in the synthesis of pharmaceutically active compounds such as neutral endopeptidase (NEP) inhibitors (see for example U.S. Pat. No. 4,722,810 and EP00509442).

The synthesis of R-biphenylalaninol has been described in WO2013/026773A1 (PCT/EP2012/066038) and is depicted in Scheme 1 hereunder.

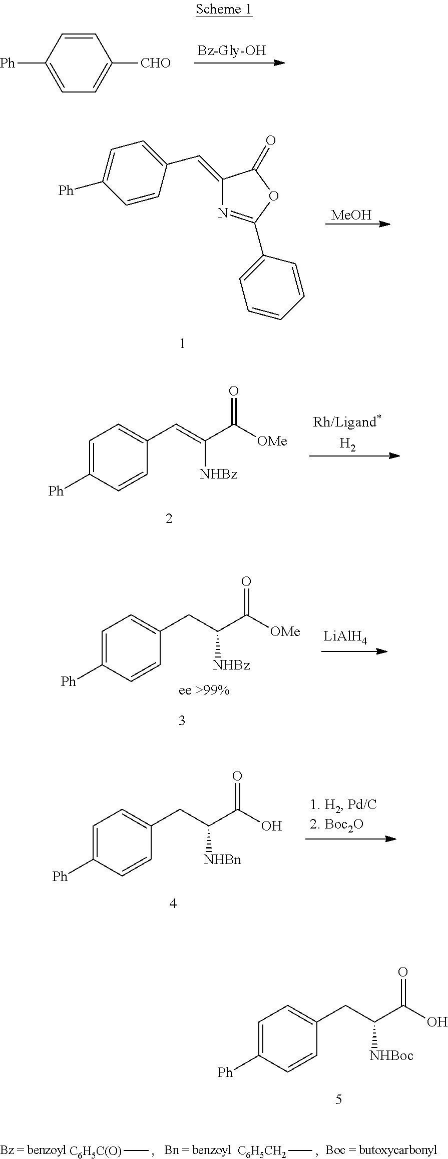

Although the synthesis as described in WO2013/026773A1 is short and economically attractive, the underlying chemical transformations comprising the simultaneous reduction of the ester and amide moiety in the intermediate 3 by lithium aluminum hydride followed by N-debenzylation go along with ecological and safety related disadvantages, comprising the handling of hazardous lithium aluminum hydride and solid aluminum waste. Furthermore, equipment suitable for hydrogen handling at high pressure is mandatory for the N-deprotection of 3.

Therefore, there is a strong need to develop inexpensive, safer and environmentally more benign methods to prepare N-Boc protected R-biphenylalaninol.

It is found that the present invention meets this objective and thus provides a process that is industrially advantageous.

### DESCRIPTION OF THE INVENTION

This invention provides methods for preparing N-Boc protected R-biphenylalaninol of formula (VI). The overall process according to the present invention is summarized in Scheme 2.

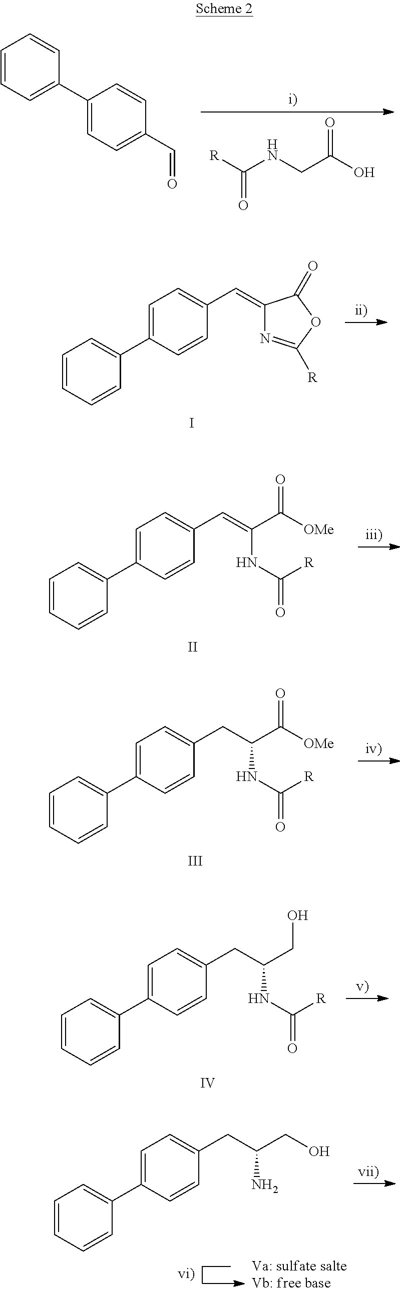

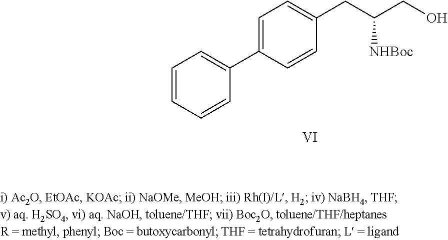 [SMILES: CC(C)(C)OC(=O)N[C@@H](CO)Cc1ccc(-c2ccccc2)cc1 ; "VI"]

The reaction sequence to the N-acyl amino acid derivatives according to formula (III) (R=Me, Ph) follows the same route as was disclosed in WO2013/026773A1, which is hereby incorporated by reference. Biphenyl formaldehyde is reacted with N-benzoylglycine and an anhydride to obtain a compound according to formula (I). By ring opening this compound is next converted into a compound according to formula (II) (R=Me, Ph). Then a compound according to formula (III) is obtained by asymmetric hydrogenation of the compound according to formula (II).

The invention now relates to a process for the manufacture of a compound according to formula (Va)

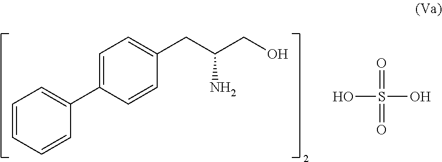

comprising
reduction of a compound according to formula (III)

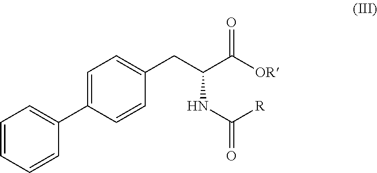

wherein R is methyl or phenyl and R′ is methyl, with a metal borohydride, resulting in an N-acyl protected R-biphenylalaninol compound according to formula (IV)

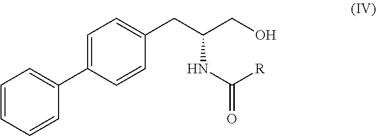

wherein R is methyl or phenyl,
and hydrolysis of this compound (IV) using sulfuric acid.

Surprisingly, the process comprising the reduction of compound (III) with metal borohydrides followed by deprotection of the N-acyl protective group now proved to be superior to the original sequence disclosed in WO2013/026773A1, in which the ester moiety was reduced together with the N-benzoyl protective group by the highly reactive lithium aluminiumhydride followed by a debenzylation reaction. The process of the present invention using the less reactive metal borohydrides instead of lithium aluminiumhydride was originally rejected as non-feasible due to the proneness for racemization of compound (III) and the harsh reaction conditions and long reaction times generally required for the N-deprotective step. However, unexpectedly, the reduction with a metal borohydride occurred under conservation of the stereoinformation in compound (III). The use of metal borohydride assures reduction of ester moiety in compound (III) without erosion of stereo-information at the neighbored stereogenic center. This is not expected due to the basic properties of a metal borohydride. The subsequent amide cleavage of compound (IV) in the presence of an aqueous sulfuric acid resulting in a compound of formula (Va) proceeded under mild conditions and short reaction times. All attempts to implement literature protocol using hydrochloric acid for the cleavage of the N-benzoyl protective group (e.g. Rozwadowska, Tetrahedron: Asymmetry 1998, 9, 1615-1618) described for very similar N-benzoyl protected starting material were not successful in our hands. The result is an easier overall process, which is safer and environmental more benign.

The process according to the present invention also offers more flexibility with regard to equipment, as hydrogenation equipment is no further needed for the N-deprotection step of compound (III). The reaction sequence comprising the reduction of N-acetyl and N-benzoyl protected phenylalanine esters with a metal borohydride followed by sulfuric acid catalyzed amide cleavage for the deprotection of the nitrogen moiety so far has been not described for the synthesis of amino alcohols derived from phenylalanine derivatives. For N-Boc instead of N-acyl protected amino alcohols a similar ester reduction is disclosed in WO2008/138561. However, as generally known, cleavage of N-Boc protecting groups is easier than cleavage of N-acyl protecting groups. Furthermore, whereas hydrolysis of phenyl amino alcohols has been disclosed to work with acids such as hydrochloric acid (e.g. Rozwadowska, Tetrahedron: Asymmetry 1998, 9, 1615-1618), hydrochloric acid mediated cleavage of bi-phenyl amino alcohols such as the N-acyl protected biphenyl alaninol system of the present invention does not work.

Surprisingly, with sulfuric acid an efficient amide cleavage for the benzoylic amino alcohol systems of the invention was obtained. Therefore, the process according to the invention offers a protocol for ester reduction in the compound according to formula (III) that replaces the use of hazardous lithium aluminum hydride with the less hazardous and cheaper sodium borohydride reagent and the subsequent amide cleavage in the presence of sulfuric acid was successful and proceeded under mild conditions and short reaction times. Furthermore, this new process allows work up of the reaction mixture without solid waste handling.

In the reduction process according to the invention, the metal borohydride can be sodium, calcium or lithium borohydride. Preferably, the metal borohydride is sodium borohydride. Optionally, the metal borohydride can be activated. Preferably, the metal borohydride is activated with a C₁-C₄ alcohol. Thus, the metal borohydride can be activated with methanol, ethanol, propanol or butanol. More preferably, the metal borohydride is activated with methanol. Most preferably, the activation of sodium borohydride is done with methanol. Activation by methanol leads to higher purity, i.e. a better chemo-selectivity for the desired compound. Moreover, cycle times required for the process are shorter. Accordingly, the present invention also relates to a process according to the invention, wherein the metal borohydride is activated with a C₁-C₄ alcohol.

Temperatures suitable for the metal borohydride mediated reduction are in the range from 10° C. to 67° C. Preferably, the temperature is higher than 10° C., more preferably above 20° C., even more preferably above 25° C. Furthermore, the temperature is preferably below 67° C., more preferably below 45° C. and even more preferably below 35° C. Most preferably, the temperature for the metal borohydride mediated reduction is in the range of 25° C. to 35° C.

The metal borohydride amount can range from 0.8 to 3.0 mol eq. to compound (III). Preferably the metal borohydride amount is in the range from 1.0 to 2.0 mol eq. to compound (III) and more preferably in the range from 1.3 to 1.5 mol eq. to compound (III).

Alcohol amounts for the activation can be varied from 2.8 to 5.6 mol eq. to compound (III), preferably in the range from 4.2 to 5.2 mol eq. More preferably, activation is done with methanol in amounts from 2.8 to 5.6 mol eq. to compound (III), most preferably in the range from 4.2 to 5.2 mol eq. to compound (III).

The reduction is complete at least 0.5 h after addition of alcohol, preferably methanol addition.

Suitable solvents for the ester reduction are alcohols, such as methanol or ethanol, chlorinated solvents such as chloromethane, or ethers such as tetrahydrofuran or mixture thereof. Preferably tetrahydrofuran is used.

The sulfuric acid mediated amide hydrolysis in the process according to the invention proved to proceed in aqueous systems under mild temperature conditions, under full retention of the stereogenic center. These mild temperature conditions represent a temperature which is above 70° C., preferably above 80° C., more preferably above 90° C., and below 110° C., preferably below 105° C., more preferably below 95° C. The invention also relates to a manufacturing process according to the invention, wherein the hydrolysis takes place at a temperature between 70° C. and 105° C.

The acid concentration for the amide hydrolysis is preferably above 30 w/w %, more preferably above 35 w/w % and most preferably above 40 w/w %. Furthermore, the acid concentration is preferably below 60 w/w %, more preferably below 55 w/w % and most preferably below 50 w/w %.

The volume of the sulfuric acid can vary from 3.0 to 8.0 L/kg starting material (IV), preferably from 3.5 to 6 L/kg starting material (IV) and more preferably from 4.0 to 5.0 L/kg starting material (IV).

Suitable solvents for the amide cleavage are aqueous systems which can contain solvents such as alcohols, such as methanol or ethanol, or ethers such as tetrahydrofuran or mixtures thereof. Preferably aqueous systems containing tetrahydrofuran are used.

The compound according to formula (V) can be used directly as the sulfate salt, i.e. the compound according to formula (Va) or after freebasing with aqueous sodium hydroxide, i.e. as the compound according to formula (Vb)

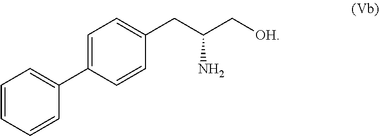 [SMILES: N[C@@H](CO)Cc1ccc(-c2ccccc2)cc1]

With freebasing we understand converting an ionic form into a free base.

The compound according to formula (Va) and/or (Vb) as obtained with the process according to the invention, can also be protected on the N-moiety. Therefore, the present invention also relates to a process according to the invention, wherein the resulting compound according to formula (Va) or (Vb) is Boc-protected to result in a compound according to formula (VI)

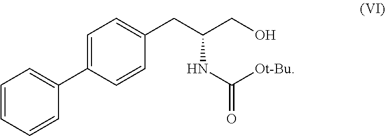 [SMILES: CC(C)(C)OC(=O)N[C@@H](CO)Cc1ccc(-c2ccccc2)cc1]

The compound according to formula (VI) can be further reacted to biaryl substituted 4-amino-butyric acid and derivatives thereof which can be further used in the production of an active pharmaceutical ingredient such as neutral endopeptidase (NEP) inhibitors as disclosed in WO2008/031567. The invention thus also relates to a process wherein the compound according to formula (VI) is further reacted to obtain an active pharmaceutical.

The novel and inventive process of the present invention proceeds via the novel and inventive intermediate compound according to formula (IV). Therefore, the present invention also relates to a compound according to formula (IV)

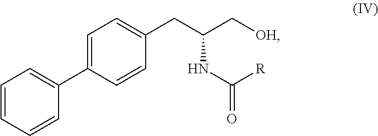

wherein R is methyl or phenyl.

Then, the product obtained via the process according to the invention is the novel and inventive compound according to formula (Va). Accordingly, the present invention also relates to a compound according to formula (Va)

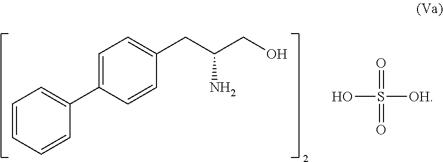

The invention further relates to all possible combinations of different embodiments and/or preferred features according to the process and intermediates according to the invention as described herein.

The invention will be elucidated with reference to the following examples, without however being restricted by these:

### EXAMPLES

### Preparation of 4-[1-Biphenyl-4-yl-meth-(Z)-ylidene]-2-phenyl-4H-oxazol-5-one

### Preparative Example According to the Prior Art A1

### Preparation of Compound (I) with R=pH

Synthesis of 4-[1-Biphenyl-4-yl-meth-(Z)-ylidene]-2-methyl-4H-oxazol-5-one I (R=Ph) by condensation of biphenyl carboxaldehyde with N-benzoyl glycine (hippuric acid)

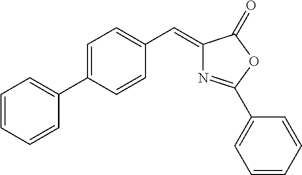 [SMILES: O=C1OC(c2ccccc2)=N/C1=C\c1ccc(-c2ccccc2)cc1]

To a dried 2500 ml reaction vessel equipped with reflux condenser and overhead stirrer were added 113 g potassium acetate (1.15 mol), ethyl acetate (1050 mL), 486 g acetic anhydride (4.77 mol), 177 g hippuric acid (0.99 mol) and 150.0 g of biphenyl formaldehyde (0.81 mol). After stirring of the resulting suspension at 60° C. for 2 h 120 ml of water were added and agitation was continued for 30 min before the suspension was cooled to room temperature and filtered. The damp product was washed with ethyl acetate and subsequently vacuum dried at max. 50° C. to obtain the title compound with a chemical purity of 97% area (retention time conforms: 11.2 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)).

### Preparation of (Z)-2-acetylamino-3-biphenyl-4-yl-acrylic acid methyl ester and (Z)-2-benzoylamino-3-biphenyl-4-yl-acrylic acid methyl ester

### Preparative Example According to the Prior Art B1

### Preparation of Compound (II) with R=Me

### Synthesis of (Z)-2-acetylamino-3-biphenyl-4-yl-acrylic acid methyl ester II (R=Me)

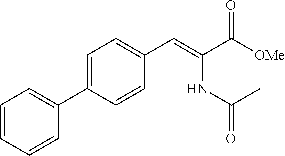 [SMILES: COC(=O)/C(=C/c1ccc(-c2ccccc2)cc1)NC(C)=O]

To a dried 250 ml reaction vessel equipped with reflux condenser and overhead stirrer were added potassium acetate (mmol), ethyl acetate (140 mL), 65 g acetic anhydride (640 mmol), 15 g N-acetyl glycine (128 mmol) and 20.0 g of biphenyl formaldehyde (109 mmol). The resulting suspension was heated to 60° C. and stirring was continued for 2 h at this temperature. After addition of 16 ml of water and additional agitation for 30 min the suspension was cooled to room temperature and filtered. The damp product was washed with ethyl acetate and subsequently vacuum dried at max. 50° C. to obtain the azlactone I (R=Me) with a chemical purity of 85% area which was immediately used in the subsequent methanolysis step (retention time: 9.0 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)).

Therefore a dried 250 ml reaction vessel equipped with reflux condenser and overhead stirrer was charged with 15 g of azlactone I (66.5 mmol) and 89 mL of methanol. After addition of sodium methylate (0.2 mol eq.) the resulting suspension was warmed to 30° C. After stirring for 2 h the reaction mixture was treated with an aqueous sodium bisulfate solution (64 mL). The resulting suspension was cooled to ambient temperature and filtered. The damp product was washed with water and subsequently vacuum dried at max 50° C. yielding the title compound.

1H NMR (200 MHz, CDCl3): δ=7.89-7.79 (m, 6H), 7.59-7.55 (m, 2H), 7.50-7.45 (m, 1H), 7.32 (s, 1H), 3.81 (s, 3H), 2.12 (s, 3H).

### Preparative Example According to the Prior Art B2

### Preparation of Compound (II) with R=pH

### Synthesis of (Z)-2-benzoylamino-3-biphenyl-4-yl-acrylic acid methyl ester II (R=Ph)

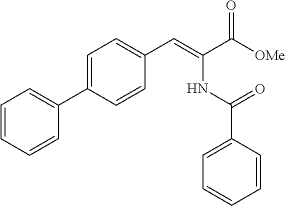 [SMILES: COC(=O)/C(=C/c1ccc(-c2ccccc2)cc1)NC(=O)c1ccccc1]

To a dried 2500 ml reaction vessel equipped with reflux condenser and overhead stirrer were added 150 g of azlactone I (R=Ph) (0.46 mol) and 760 mL of methanol. After addition of sodium methylate (0.1 mol eq.) the resulting suspension was warmed to 30° C. After stirring for 2 h acetic acid was added (0.2 mol eq.) followed by addition of water (450 mL). The resulting suspension was cooled to ambient temperature and filtered. The damp product was washed with water and subsequently vacuum dried at max 50° C. yielding the title compound with a chemical purity of 99.7% area (retention time conforms 7.4 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)).

### Preparative Example According to the Prior Art C1

### Preparation of Compound (III) with R=Me

### Asymmetric Hydrogenation of (Z)-2-acetylamino-3-biphenyl-4-yl-acrylic acid methyl ester II (R=Me)

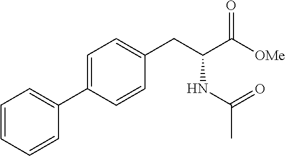 [SMILES: COC(=O)[C@@H](Cc1ccc(-c2ccccc2)cc1)NC(C)=O]

The catalyst suspension was prepared from bis(1,5-cyclooctadiene) rhodium(I)tetrafluoroborate (0.09 mmol) and (S)-1-(dinaphto[2,1-d:1′,2′-f][1,3,2]dioxaphosphepin-4-yl)piperidine (S-PiPhos) (0.19 mmol) in 132 mL THF (tetrahydrofuran) under inert reaction conditions which can be achieved by having an atmosphere of nitrogen or argon. To this solution was added 10.0 g N-acetyl dehydroamino acid methyl ester II (34 mmol). The thus obtained mixture was hydrogenated (10 bar H2; 22-28° C.) until full conversion was reached after 16 h (based on HPLC) providing compound III after removal of THF in vacuo (retention time 5.8 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)).

1H NMR (200 MHz, CDCl3): δ=7.58-7.50 (m, 4H), 7.45-7.40 (m, 2H), 7.36-7.33 (m, 1H), 7.17-7.15 (m, 2H), 6.07 (d, J=5 Hz, 1H), 4.95-4.89 (m, 1H), 3.74 (s, 3H), 3.23-3.09 (m, 2H), 1.99 (s, 3H).

### Preparative Example According to the Prior Art C2a

### Preparation of Compound (III) with R=pH and Catalyst in DCM

### Asymmetric Hydrogenation of (Z)-2-Benzoylamino-3-Biphenyl-4-Yl-acrylic acid methyl ester to III (R=Ph)

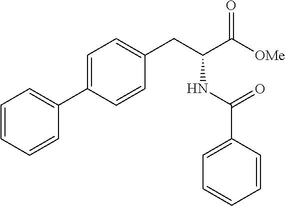 [SMILES: COC(=O)[C@@H](Cc1ccc(-c2ccccc2)cc1)NC(=O)c1ccccc1]

The catalyst solution was prepared from bis(1,5-cyclooctadiene) rhodium(I)tetrafluoroborate (45 mg; 0.11 mmol) and (S)-1-(dinaphto[2,1-d:1′,2′-f][1,3,2]dioxaphosphepin-4-yl)piperidine (S-PiPhos) (94 mg; 0.24 mmol) in 1.4 mL DCM under inert reaction conditions which can be achieved by having an atmosphere of nitrogen or argon. This solution was added to a solution of 80.0 g N-benzoyl dehydroamino acid methyl ester II (224 mmol) in 265 ml of THF. The thus obtained mixture was hydrogenated (5 bar H2; 22-28° C.) until full conversion was reached after 4 h (based on HPLC) providing compound III with a chemical purity of 100% area (retention time conforms 7.7 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)) and an optical purity of 99.5% ee (Chiralpak IC-3, Fa. Daicel, 150×4.6 mm, 3 μm, Water+0.1 Vol. % Diethylamine, 40% Acetonitril+0.1 Vol. % Diethylamine)

### Preparative Example According to the Prior Art C2b

### Preparation of Compound (III) with R=pH and Catalyst in THF

### Asymmetric Hydrogenation of (Z)-2-benzoylamino-3-biphenyl-4-yl-acrylic acid methyl ester II (R=Ph) with catalyst suspension in THF

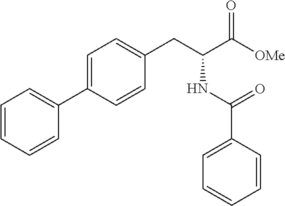 [SMILES: COC(=O)[C@@H](Cc1ccc(-c2ccccc2)cc1)NC(=O)c1ccccc1]

The catalyst suspension was prepared from bis(1,5-cyclooctadiene) rhodium(I)tetrafluoroborate (45 mg; 0.11 mmol), (S)-1-(dinaphto[2,1-d:1′,2′-f][1,3,2]dioxaphosphepin-4-yl)piperidine (S-PiPhos) (94 mg; 0.24 mmol) and 1.4 mL THF under inert reaction conditions which can be achieved by having an atmosphere of nitrogen or argon. This suspension was added to a solution of 80.0 g N-benzoyl dehydroamino acid methyl ester II (R=Ph) (224 mmol) in 265 ml of THF. The thus obtained mixture was hydrogenated (5 bar H2; 22-28° C.) until full conversion was reached after 4 h (based on HPLC) providing compound III with a chemical purity of 100% area (retention time conforms 7.7 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)). and an optical purity of 99.5% ee (Chiralpak IC-3, Fa. Daicel, 150×4.6 mm, 3 μm, Water+0.1 vol. % diethylamine, 40% acetonitril+0.1 vol % diethylamine)

### Preparative Example According to the Prior Art C2c

### Preparation of Compound (III) with R=pH and Catalyst in DCM, Different Substrate to Catalyst Ratios

Asymmetric Hydrogenation of (Z)-2-benzoylamino-3-biphenyl-4-yl-acrylic acid methyl ester II (R=Ph) with Catalyst Solution in DCM

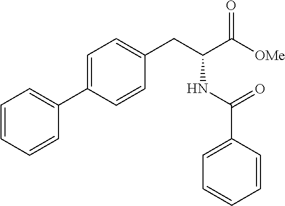 [SMILES: COC(=O)[C@@H](Cc1ccc(-c2ccccc2)cc1)NC(=O)c1ccccc1]

The catalyst solution was prepared from bis(1,5-cyclooctadiene) rhodium(I)tetrafluoroborate (36 mg; 0.09 mmol), (S)-1-(dinaphto[2,1-d:1′,2′-f][1,3,2]dioxaphosphepin-4-yl)piperidine (S-PiPhos) (75 mg; 0.19 mmol) and 1 mL DCM under inert reaction conditions which can be achieved by having an atmosphere of nitrogen or argon. This solution was added to a solution of 80.0 g N-benzoyl dehydroamino acid methyl ester II (224 mmol) in 265 ml of THF. The thus obtained mixture was hydrogenated (5.5 bar H2; 22-28° C.) until full conversion was reached after 4 h (based on HPLC) providing compound III with a chemical purity of 100% area (retention time conforms: 7.7 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)) and an optical purity of 99.5% ee (Chiralpak IC-3, Fa. Daicel, 150×4.6 mm, 3 μm, Water+0.1 vol. % diethylamine, 40% acetonitril+0.1 vol % diethylamine)

### Reduction of (R)-2-Benzoylamino-3-Biphenyl-4-Yl-Propionic Acid Methyl Ester with Sodium Borohydride in the Presence of Methanol (III-4 IV)

### Example 1

### Preparation of Compound (IV) with R=pH

### Sodium Borohydride Activation by Dosage of Methanol to Sodium Borohydride

To a dried 250 ml reaction vessel equipped with reflux condenser and overhead stirrer were added 217 mL of a THF (tetrahydrofuran) solution comprising (R)-2-Benzoylamino-3-biphenyl-4-yl-propionic acid methyl ester (40, 2, 112 mmol) and sodium borohydride (5.9 g, 156 mmol) followed by dosage of methanol (MeOH)* (20.1 g, 290 mmol). The reaction was subsequently heated to 40° C. and stirred for 3 h. The obtained mass was quenched with THF (13.5 mL) and water (70 mL). After phase separation and extraction of the aqueous phase with THF the combined organic phases were washed with a concentrated sodium chloride solution. After removal of the aqueous phase the organic phase was concentrated in vacuo to yield the corresponding N-benzoyl protected amino alcohol with a chemical purity of 99.4% area (retention time conforms 6.0 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)) and an optical purity of 98% ee (Chiralpak IC-3, Fa. Daicel, 150×4.6 mm, 3 μm, Water+0.1 Vol. % diethylamine, 40% acetonitril+0.1 Vol. % diethylamine) *Same results will be achieved when reaction is performed in the presence of ethanol, propanol and butanol

### Example 2

### Preparation of Compound (IV) with R=pH

### Sodium Borohydride Activation by Dosage of Methanol to Sodium Borohydride—Reduced NaBH4 and MeOH Amount

To a dried 250 ml reaction vessel equipped with reflux condenser and overhead stirrer were added 109 mL of a THF solution comprising (R)-2-Benzoylamino-3-biphenyl-4-yl-propionic acid methyl ester (20.0 g, 55.6 mmol) and sodium borohydride (2.8 g, 74 mmol) followed by dosage of MeOH* (9.3 g, 290 mmol). The reaction was subsequently heated to 30° C. and stirred for 2 h. The obtained mass was quenched with THF (13.5 mL) and water (70 mL). After phase separation and extraction of the aqueous phase with THF the combined organic phases were washed with a concentrated sodium chloride solution. After removal of the aqueous phase the organic phase was concentrated in vacuo to yield the corresponding N-benzoyl protected amino alcohol with 95% yield and a chemical purity of 99.3% area (retention time conforms 6.0 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)), and an optical purity of 98% ee (Chiralpak IC-3, Fa. Daicel, 150×4.6 mm, 3 μm, Water+0.1 Vol. % diethylamine, 40% acetonitril+0.1 Vol. % diethylamine) * Same results will be achieved when reaction is performed in the presence of ethanol, propanol and butanol

### Example 3

### Preparation of Compound (IV) with R=pH

### Sodium Borohydride Activation by Dosage of Methanol to Sodium Borohydride—Further Reduced NaBH4 and MeOH Amount

1.05 g (1.0 eq) NaBH4 were suspended in 100 ml THF under inert reaction conditions which can be achieved by having an atmosphere of nitrogen or argon in a 4 necked round bottom flask equipped with an overhead stirrer, a reflux condenser and a dropping funnel. 10 g of 2-Benzoylamino-3-biphenyl-4-yl-propionic acid methyl ester are added solid, an almost clear yellowish solution was formed. The reaction mixture is heated to slight reflux (95° C.) 3.57 g (4 eq) methanol* were added over 15 min. The reaction mixture was aged at reflux until HPLC shows complete conversion (ca. 2 h). Then the reaction mixture was cooled to rt and 60 ml water are added. After 30 min aging the layers were separated and the aqueous layer was extracted with 30 ml THF. The combined organic layers were washed with 60 ml half-saturated sodium bicarbonate and 60 ml half-saturated brine. The resulting organic solution (ca. 60 ml) was slowly dripped onto 60 ml water at rt over at least 1 h. A nice, stirrable suspension forms. Ca. 30 ml THF were distilled off under vacuum at max. 30° C. A thick but still stirrable suspension forms. The product was isolated on a filter nutsch and washed portion wise with 40 ml water and dried in vacuo at 45° C. to yield 7.79 g (84.5%). * Same results will be achieved when reaction is performed in the presence of ethanol, propanol and butanol

### Example 4

### Preparation of Compound (IV) with R=pH

### Sodium Borohydride Activation by Dosage of Sodium Borohydride to a MeOH Containing Solution of III in THF

To a dried 250 ml reaction vessel equipped with reflux condenser and overhead stirrer were added 126 mL of a THF solution comprising (R)-2-Benzoylamino-3-biphenyl-4-yl-propionic acid methyl ester (29.6 g, 82.6 mmol) and sodium borohydride (4.4 g, 117.5 mmol) followed by dosage of MeOH* (7.5 g, 235 mmol). The reaction was subsequently heated to 30° C. and stirred for 16 h. The obtained mass was quenched with THF (17 mL) and water (78 mL). After phase separation and extraction of the aqueous phase with THF the combined organic phases were washed with a concentrated sodium chloride solution. After removal of the aqueous phase the organic phase was concentrated in vacuo to yield the corresponding N-benzoyl protected amino alcohol with 99% yield and a chemical purity of 98.7% area (retention time conforms 6.0 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)) and an optical purity of 97% ee (Chiralpak IC-3, Fa. Daicel, 150×4.6 mm, 3 μm, Water+0.1 Vol. % diethylamine, 40% acetonitril+0.1 Vol. % diethylamine) * Same results will be achieved when reaction is performed in the presence of ethanol, propanol and butanol

### Reduction of N—((R)-2-Biphenyl-4-Yl-1-Hydroxymethyl-Ethyl)Acetamide with Sodium Borohydride in the Presence of Methanol

### Example 5

### Preparation of Compound (IV) with R=Me

### Sodium Borohydride Activation by Dosage of Methanol to a NaBH4

To a dried 250 ml reaction vessel equipped with reflux condenser and overhead stirrer were added 132 mL of a THF solution comprising N—((R)-2-Biphenyl-4-yl-1-hydroxymethyl-ethyl)-acetamide (34 mmol) and sodium borohydride (55 mmol) followed by dosage of MeOH* (145 mmol). The reaction was subsequently heated to 30° C. and stirred for 2 h. In process control revealed incomplete conversion followed by additional 4 h reaction time. The obtained mass was quenched with water (50 mL) and THF (35 mL). After phase separation and extraction of the aqueous phase with THF the combined organic phases were washed with a concentrated sodium chloride solution. After removal of the aqueous phase the organic phase was concentrated in vacuo to yield the corresponding N-acetyl protected amino alcohol.

(retention time 4.2 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)).

1H NMR (200 MHz, CDCl3): δ=7.58-7.53 (m, 4H), 7.45-7.41 (m, 2H), 7.30-7.26 (m, 3H), 5.76 (m, 1H), 4.23-4.18 (m, 1H), 3.74-3.60 (m, 2H), 2.93-2.91 (m, 2H), 1.98 (s, 3H). * Same results will be achieved when reaction is performed in the presence of ethanol, propanol and butanol

### Reduction of N—((R)-2-Biphenyl-4-yl-1-hydroxymethyl-ethyl)-acetamide with lithium borohydride

### Example 6

### Preparation of Compound (IV) with R=pH

Sodium borohydride activation by lithium chloride as the corresponding lithium salt To a dried 250 ml reaction vessel equipped with reflux condenser and overhead stirrer were added a THF solution comprising (R)-2-Benzoylamino-3-biphenyl-4-yl-propionic acid methyl ester (1 moleq.) and sodium borohydride (1.5 moleq.) followed by dosage of lithium chloride (1.5 moleq. g). The reaction was subsequently heated to 65° C. and stirred for 29 h. The obtained mass was quenched with THF and water. After phase separation and extraction of the aqueous phase with THF the combined organic phases were treated with water for crystallization of the title compound which was isolated with a chemical purity of 95% area.

### Sulphuric Acid Mediated Amide Cleavage of N-Benzoyl Protected Amino Alcohol IV to the Biphenylalaninol V

### Example 7

### Preparation of Compound (Vb)

### Amide Cleavage and Isolation of Biphenylalaninol as it's Free Base

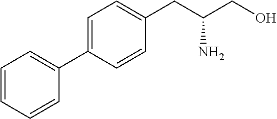 [SMILES: N[C@@H](CO)Cc1ccc(-c2ccccc2)cc1]

10.0 g IV (30.8 mmol) were suspended in 80 mL 6 M sulfuric acid (H2SO4) under inert reaction conditions which can be achieved by having an atmosphere of nitrogen or argon in a 500 ml 4 necked round bottom flask equipped with an overhead stirrer, a reflux condenser and a dropping funnel. The reaction mixture was heated to slight reflux (95° C.). The reaction mixture was aged at 95° C. for 20 h. The reaction mixture was cooled to room temperature and the pH was adjusted to 10-11 with 20% NaOH (161 ml; pH=11.1). The reaction mixture was stirred at pH=11 for 2 h. Then the suspension was filtered and the filter cake was washed portion wise with a total of 40 ml 1 N NaOH and portion wise with a total of 120 ml water and dried in vacuo at 45° C. to yield 6.56 g (95.7%) of the amino alcohol with a chemical purity of 98% area.

### Example 8

### Preparation of Compound (Va)

### Amide Cleavage and Isolation of Biphenylalaninol as it's Sulfate Salt Followed by Telescoping into the Synthesis of N-Boc Protected R-Biphenylalaninol

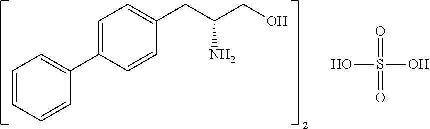

29.4 g IV (88.7 mmol) were suspended in 146.5 g of a 49% H2SO4 under inert reaction conditions which can be achieved by having an atmosphere of nitrogen or argon in a 250 mL 4 necked Schmizo reactor flask equipped with an overhead stirrer, a reflux condenser. The reaction mixture was heated to reflux (95-105° C.). The reaction mixture was aged at 95-105° C. for 16 h. After cooling to room temperature, the suspension was filtered and washed with water yielding the title compound with a chemical purity of 95% area (retention time conforms 2.3 min; Poroshell 120 C-18, Fa. Agilent, 100×3.0 mm, 0.1 vol % aq. trifluoro acetic acid solution, 0.1 vol % trifluoroacetic acid acetonitrile solution, −5 min (70:30), 0 min (70:30), 10 min (10:90), 15 min (10:90)) Optical purity was determined after derivatisation to the N-Boc protected amino alcohol to be 99% ee (Chiralpak IC-3, Fa. Daicel, 150×4.6 mm, 3 μm, Water+0.1 Vol. % diethylamine, 40% acetonitril+0.1 Vol. % diethylamine).

1. A process for the manufacture of a compound according to formula (Va) 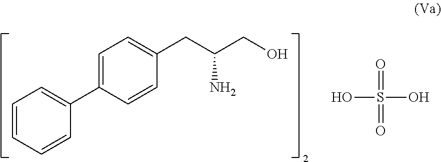
comprising
reduction of a compound according to formula (III) 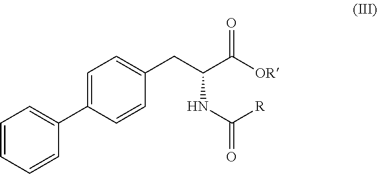
wherein R is methyl or phenyl and R′ is methyl, with a metal borohydride, resulting in an N-acyl protected R-biphenylalaninol compound according to formula (IV) 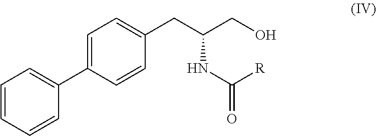
wherein R is methyl or phenyl,
and hydrolysis of this compound (IV) using sulfuric acid.

2. The process according to claim 1, wherein the metal borohydride is sodium borohydride.

3. The process according to claim 1, wherein the metal borohydride is activated by a C₁-C₄ alcohol.

4. The process according to claim 3, wherein the metal borohydride is activated by methanol.

5. The process according to claim 1, wherein the hydrolysis takes place at a temperature between 70° C. and 105° C.

6. The process according to claim 1, wherein the resulting compound according to formula (Va) is subjected to freebasing to obtain a compound according to formula (Vb)

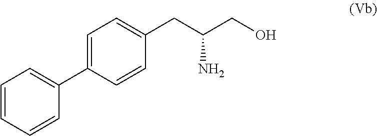 [SMILES: N[C@@H](CO)Cc1ccc(-c2ccccc2)cc1]

7. The process according to claim 1, wherein the resulting compound according to formula (Va) or (Vb) is Boc-protected to give a compound according to formula (VI)

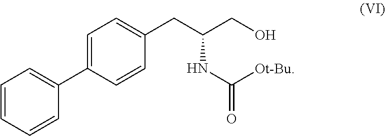 [SMILES: CC(C)(C)OC(=O)N[C@@H](CO)Cc1ccc(-c2ccccc2)cc1]

8. The process according to claim 7, wherein the compound according to formula (VI) is further reacted to obtain an active pharmaceutical.

9. An N-Acyl protected biphenylalaninol compound according to formula (IV) 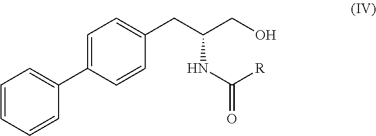
wherein R is methyl or phenyl.

10. A compound according to formula (Va)

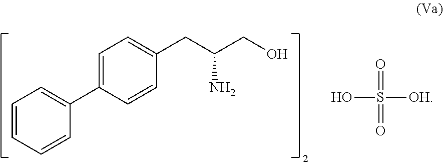

11. The process according to claim 6, wherein the resulting compound according to formula (Va) or (Vb) is Boc-protected to give a compound according to formula (VI)

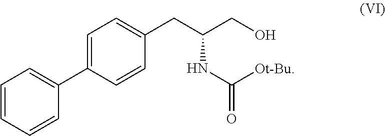 [SMILES: CC(C)(C)OC(=O)N[C@@H](CO)Cc1ccc(-c2ccccc2)cc1]

12. The process according to claim 11, wherein the compound according to formula (VI) is further reacted to obtain an active pharmaceutical.
